(19) United States
(12) Patent Application Publication
(10) Pub. No.: US 2025/0114045 A1
(43) Pub. Date: Apr. 10, 2025

(54) Systems and Methods for Assessing Hemodynamic Variability

(71) Applicant: The Board of Trustees of the Leland Stanford Junior University, Stanford, CA (US)

(72) Inventors: Todd P. Coleman, Stanford, CA (US); Andrew Sho Perley, Stanford, CA (US); Sabrina Liu, Stanford, CA (US)

(73) Assignee: The Board of Trustees of the Leland Stanford Junior University, Stanford, CA (US)

(21) Appl. No.: 18/908,631

(22) Filed: Oct. 7, 2024

Related U.S. Application Data
(60) Provisional application No. 63/588,566, filed on Oct. 6, 2023.

(51) Int. Cl. A61B 5/00; A61B 5/024; A61B 5/28; A61B 5/346

(52) U.S. Cl. CPC A61B 5/7275; A61B 5/02405; A61B 5/28; A61B 5/346; A61B 5/6852; A61B 5/7267; A61B 2560/0462; A61B 2562/0247

(57) ABSTRACT

Systems and methods for computing variation in physiological parameters and covariate relationships are provided. Sensor data derived can be transformed into physiological interval data. A point-process model with global optimization can be fit to the physiological interval data. Variation of the physiological interval data can be computed using the fitted model. Covariate data can also be collected. A state-space model with global optimization can be fit to the physiological interval data and covariate data.

### CROSS REFERENCE TO RELATED APPLICATIONS

This application claims benefit of U.S. Provisional Patent Application No. 63/588,566, filed Oct. 6, 2023, entitled “Systems and Methods for Assessing Hemodynamic Variability,” the disclosure of which is hereby incorporated by reference in its entirety for all purposes.

### TECHNOLOGICAL FIELD

The disclosure is generally directed to systems and methods for monitoring hemodynamic data, and more specifically for assessing variability in hemodynamic data, such as heart rate variability.

### BACKGROUND

Heart rate variability (HRV) is the variation of the time interval between heartbeats over a period of time. Provided in FIG. 1A is an example of an electrocardiogram (ECG) showing a wave of heartbeats over a period time. An ECG wave has three main components: the P wave, the QRS complex, and the T wave. The P wave represents atrial depolarization, where the right and left atria of the heart contract representing blood transferring into the right and left ventricles. The QRS complex represents ventricular depolarization, where the right and left ventricles of the heart contract, representing blood transferring blood from the ventricles into the lungs and throughout the body. The T wave represents ventricular repolarization. A heartbeat cycle can be defined between any the components or other features of the ECG wave. A common definition is between the R peaks, referred to as the RR interval. As can be seen in FIG. 1A, the length of time between RR intervals varies, depicting the variation of the time interval between heartbeats. For more on heart rate variability, see U. Rajendra Acharya, et al., Med Biol Eng Comput. 2006, 44 (12): 1031-1051.

### SUMMARY

In some aspects, the techniques described herein relate to a computer system, including: a computational processing system; and memory including an application for fitting a point-process model with global optimization; wherein the application directs the computational processing system to: receive physiological interval data; fit the point-process model with global optimization to the physiological interval data; and compute variability of the physiological interval data using the fitted point-process model with global optimization.

In some aspects, the techniques described herein relate to a system, wherein the physiological interval data is heartbeat interval data.

In some aspects, the techniques described herein relate to a system, wherein the computer system is a part of a health monitoring system that includes a sensor configured to capture physiological interval data, wherein the application directs the computational processing system to: sense physiological parameters of a patient to yield the physiological interval data.

In some aspects, the techniques described herein relate to a system, wherein the application directs the computational processing system to perform the following real-time: sense physiological parameters of a patient to yield the physiological interval data; receive physiological interval data; fit the point-process model with global optimization to the physiological interval data; and compute variability of the physiological interval data using the fitted point-process model with global optimization.

In some aspects, the techniques described herein relate to a system, wherein the sensor is in connection with and the computational processing system is housed within a wearable device, a hemodynamic monitoring system, or an electrocardiography device.

In some aspects, the techniques described herein relate to a system, wherein the sensor is one of: one or more leads of an electrocardiography machine or ECG-like device, a blood pressure transducer catheter, a blood pressure cuff, an ultrasound transducer, a magnetic resonance imaging scanner, or a photoplethysmograph.

In some aspects, the techniques described herein relate to a system, wherein the application directs the computational processing system to: apply a distribution onto the physiological interval data, wherein the distribution yields a global optimization when utilized with the point-process model.

In some aspects, the techniques described herein relate to a system, wherein the distribution is a gamma distribution.

In some aspects, the techniques described herein relate to a system, wherein the point-process model with global optimization includes a regression model.

In some aspects, the techniques described herein relate to a system, wherein the regression model is a generalized linear model.

In some aspects, the techniques described herein relate to a system, wherein the application directs the computational processing system to: infer weights for the physiological interval data.

In some aspects, the techniques described herein relate to a system, wherein the weights are unconstrained and capable of being negative or positive.

In some aspects, the techniques described herein relate to a system, wherein the application directs the computational processing system to: infer a shape parameter from the weights.

In some aspects, the techniques described herein relate to a system, wherein the memory including an application for training a machine-learning model; wherein the application for training a machine-learning model directs the computational processing system to: train a machine-learning model to predict a biological characteristic from weights inferred for the physiological interval data; wherein the data for training the model includes inferred weights from at least two cohorts such that the machine-learning model is trained to predict the biological characteristic; wherein the biological characteristic is associated with one of cohort of the at least two cohorts.

In some aspects, the techniques described herein relate to a system, wherein the machine-learning model is to train to predict a likelihood or presence of a medical disorder selected from: orthostatic hypotension, postprandial hypotension, multiple system atrophy, pure autonomic failure, afferent baroreflex failure, familial dysautonomia, heart failure, metabolic disorder, diabetes, or cardiovascular disease.

In some aspects, the techniques described herein relate to a system, wherein the memory including an application for fitting a state-space model with global optimization, wherein the application for fitting a state-space model with global optimization directs the computational processing system to: receive covariate data; fit the state-space model with global optimization to the physiological interval data and the covariate data; and compute a relationship between the physiological interval data and the covariate data using the fitted state-space model with global optimization.

In some aspects, the techniques described herein relate to a system, wherein the covariate data is related to the autonomic nervous system.

In some aspects, the techniques described herein relate to a system, wherein the covariate data includes at least one of: respiration, sleep, stress, posture, inflammation, or drugs.

In some aspects, the techniques described herein relate to a system, wherein the state-space model includes a Gauss-Markov process.

In some aspects, the techniques described herein relate to a system, wherein the state-space model includes a high dimensionality solution with an alternating direction method of multipliers.

### BRIEF DESCRIPTION OF THE DRAWINGS

The description and claims will be more fully understood with reference to the following figures and data graphs, which are presented as examples of the disclosure and should not be construed as a complete recitation of the scope of the disclosure.

FIG. 1A provides a schematic of an example of an electrocardiogram waveform.

FIG. 1B provides a schematic of the relationship between effectors on autonomic state and computational assessments on these relationships.

FIG. 2A provides an example of a method to fit a point-process model with global optimization to heartbeat interval data and compute heart rate variability.

FIG. 2B provides an example of a method to fit a state-space model with global optimization to heartbeat interval data and covariate data, and compute relationships between the heart rate variability and covariates.

FIG. 2C provides an example of a method to train a machine-learning model to assess biological characteristics associated with heart rate variation.

FIG. 3 provides an example of a computational system or a health monitoring system.

FIG. 4 provides Kolmogorow-Smirnov plots of time-rescaled ISIs for different numbers of RR intervals fit. In each plot from left to right an increasing number of RR intervals was used to fit both the Inverse-Gaussian and Gamma models. The time-rescaling procedure was used which in principle renders its outputs to be unit-rate independent exponentially distributed. The dotted lines are 95% confidence intervals, and the 45-degree diagonal line represents perfect model fit. Notice that as the number of RR intervals fit increases, the Gamma model is closer to the center line and achieves better model fit. This suggests that while the Gamma model might suffer from worse fit with less data, as the amount of data increases, the Gamma model outperforms the Inverse-Gaussian model.

FIG. 5 provides plot of performance metrics comparing the Inverse-Gaussian to Gamma model. The runtime for each algorithm is plotted in upper box. For all sets of RR intervals fit, the Gamma model ran, on average, 4 times faster. The KS distance (comparing model fit) for each model is plotted in lower box. While the Gamma model has larger KS distance values for small amounts of data, for larger amounts of data the Gamma model has a lower KS distance and thus has better model fit. This confirms results in FIG. 4.

FIG. 6 provides a schematic of a state-space model for heartbeat dynamics. yₙ is the observed nth RR interval. xₙ is the nth latent vector of weights that determines the affine combination relating the past d intervals to the current one.

FIG. 7 provides from top to bottom, URR estimates from \hat{ }x (solid line) taken at ADMM iterations 10, 100, and 659 when the procedure terminates for subject 12726. They are compared to the observed RR intervals (stars). As the procedure progresses, μ_{RR} is less overfit to the data. This is reflected in the associated KS plots on the right where the center, 45-degree diagonal line is if the model fit perfectly with the 95% confidence intervals depicted in dashed-dotted lines.

FIG. 8 provides plots of heart rate for subject 12726 with μ_{HR} estimated using our model (middle) and local averaging (bottom) in response to tilt table angles (top). Our model reflects a more sudden change in heart rate near the rapid tilt down that is less apparent than when computed with local averaging.

FIG. 9 provides a table depicting results of the gamma GLM+GLP model in reacting to changes from tilt-table assessment.

### DETAILED DESCRIPTION

The current disclosure details systems and methods to compute variation of physiological parameter data with high temporal resolution. In many embodiments, physiological parameter data is captured utilizing a sensor and assessed for variation with high temporal resolution. In some embodiments, parameter data variation is computed using a processor system of a computer or other computational device. In some embodiments, parameter data variation is computed utilizing a health monitoring system, which can comprise a processor system for performing computation. In some embodiments, the parameter data is assessed retroactively, after data is captured. In some embodiments, the parameter data can be captured via sensors of a healthy monitoring system and further assessed for variation by the health monitoring system in real time. In some embodiments, the parameter data is physiological interval data, such as (for example) heartbeat intervals. Physiological interval data can be captured or derived from a variety of sensors, such as (for example) electrode leads for electrocardiography (ECG) or photodetectors for photoplethysmography (PPG). In many embodiments, variation of heartbeat intervals is computed with high temporal resolution, and the results of the heartbeat variation is used to assess the health of an individual, especially health characteristics related to the autonomic nervous system.

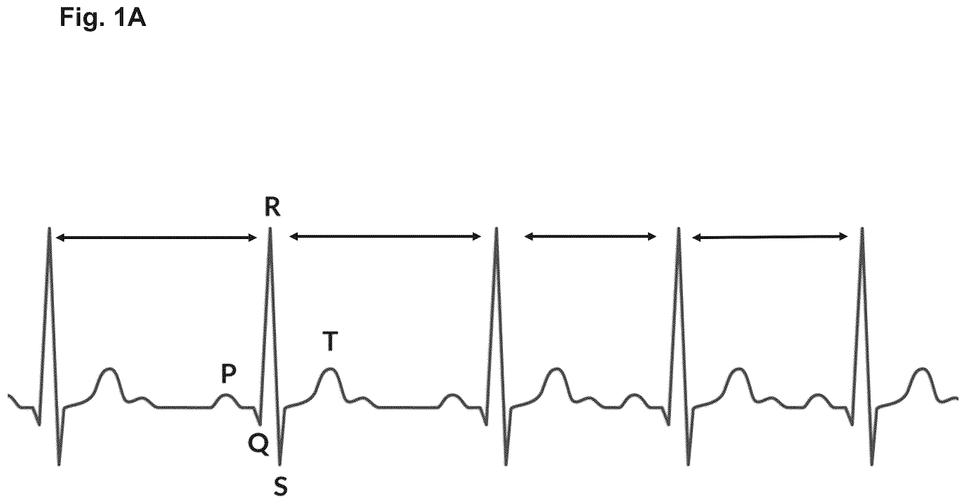

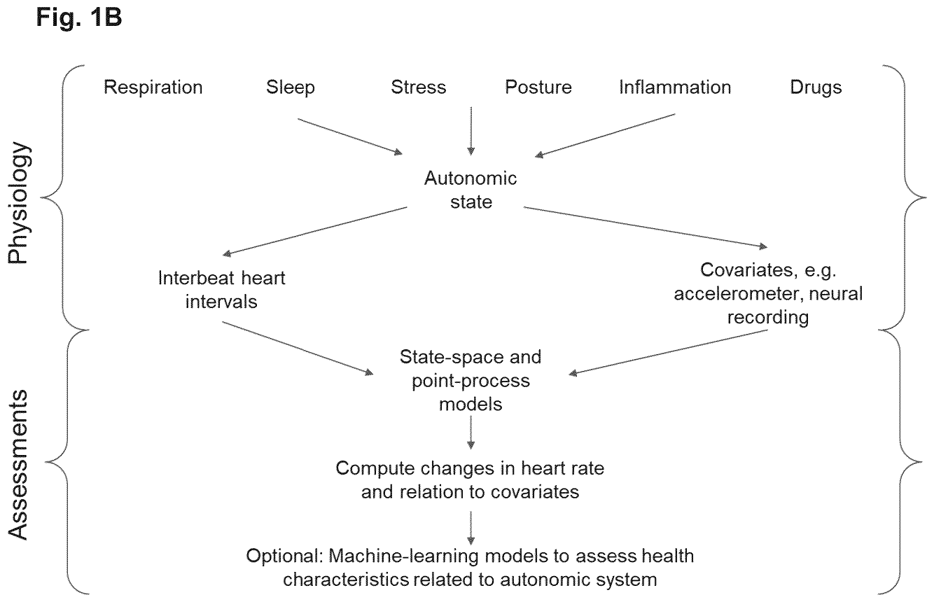

Provided in FIG. 1B is a flow chart of the physiology and assessments of such physiology. The autonomic state comprises the sympathetic and parasympathetic nervous systems. The sympathetic nervous system is the part of the nervous system that signals for activated “fight-or-flight” response. By contrast, the parasympathetic nervous system is the part of the nervous system that signals for relaxing and conservation of energy for a “rest-and-digest” response. These autonomic systems are directly influenced by many factors, such as (for example) respiration, sleep, stress, posture, inflammation, and drugs.

The autonomic state can be assessed by monitoring the physiological parameters that directly involved, such as heart rate. Heart rate can be monitored by a number of sensor systems, especially hemodynamic monitoring systems that monitor cardiovascular function. Examples of these monitoring systems include electrocardiograms, vascular plethysmograms, blood pressure sensors, blood flow sensors, etc. In addition to monitoring of physiological heart rate, covariates can also be monitored, which are variables that covary with HRV. Many covariates are biological processes involved with the autonomic state. For example, respiratory rate can be monitored via capnography, sleep and posture can be monitored via accelerometers, and neural activity can be monitored via neural recording devices.

In several embodiments described herein, point-process models are utilized to assess heartbeat intervals for variation and state-space models (FIG. 1B). And in some embodiments, a state-space model is utilized to assess the relationship of a covariate on heart rate variation. These models can compute these assessments retroactively or in real time, as may be useful depending on the application. For example, a retroactive assessment can be useful when trying to diagnose an underlying health condition that is affecting the autonomic state. Alternatively, a real-time assessment can be useful to monitor heart rate and covariates to ensure positive health outcomes during a procedure, such as a surgical procedure.

Machine-learning models can optionally be trained to use the outputs generated by one or both of the point-process and state-space models to assess and diagnose health characteristics related to autonomic system (FIG. 1B). For example, various autonomic system related health disorders will have an influence on HRV and/or covariates, resulting in a difference between healthy individuals and affected individuals. A machine-learning model can be trained to process the differences of HRV and/or covariate signals to predict whether a particular individual is healthy or affected by a health disorder. The machine-learning model output can be used to make a diagnosis and determine an appropriate treatment.

Traditional approaches to determine HRV rely on temporal and spectral windowing and smoothing approaches (see Task Force of the European Society of Cardiology and the North American Society of Pacing and Electrophysiology. Eur Heart J. 1996, 17 (3): 354-381, the disclosure of which is hereby incorporated by reference. Smoothing does not achieve true instantaneous temporal resolution because they do not consider the point-process nature of heartbeat generation. Heartbeats are not iteratively generated in a regular pattern, but instead are discrete events that occur in continuous time because they are generated via biological and physiological responses that are irregular.

Although some computational models have been generated to better capture temporal resolution, these models can be slow, computationally inefficient, and require expert handling, rendering them unpractical in many real-world applications. One example is a point-process history-dependent inverse Gaussian (HDIG) model developed by R. Barbieri, et al. (Am J Physiol Heart Circ Physiol. 2005, 288(1): H424-H435, the disclosure of which is hereby incorporated by reference). In this approach, the membrane potential of cardiac pacemaker cells is modeled as a Gaussian random walk with linear drift, and in doing so, an inverse Gaussian model is hypothesized for the interval between successive heartbeats such as the RR interval. This model, however, fails to determine a global optimum and thus requires an expert's judicious selection of initial conditions for model fitting, which is also results in the model being computationally inefficient. These models also lack interpretability due to the fact that variable weights are constrained to a selection of predetermined values.

Here, novel systems and methods are described that utilize a framework to fit a point-process model to capture high resolution of heartbeat dynamics. In several embodiments, the framework computes a global optimum, yields greater computational efficiency, and does not require expert selection of initial conditions. In many embodiments, the framework estimates a convex or concave maximum likelihood to yield a global optimum. In several embodiments, the framework comprises a regression model (e.g., generalized linear model (GLM)). In many embodiments, the data is applied to a distribution that yields an optimal solution. In some embodiments, the data is applied to a Gamma distribution. In many embodiments, the framework can be utilized to generate a diagnostic for autonomic system related health characteristics. In many embodiments, the framework can be utilized in real-time to provide high temporal resolution of HRV, allowing it to be utilized in various hemodynamic monitoring systems and wearable device systems. Analysis of the framework via the Kolmogorov-Smirnov (KS) distance revealed that a convex point-process model incorporating a Gamma GLM outperformed the inverse Gaussian model developed by R. Barbieri, et al. in goodness of fit. The analysis also revealed that when high amount of input data is utilized, the convex point-process model incorporating a Gamma GLM improved the runtime. And when assessing covariate relationship, a state-space model based on the Gamma GLM was able to more quickly and accurately identify covariate influence on HRV than local averaging techniques.

Based on these assessment results and in accordance with several embodiments of the disclosure, a computational framework comprises a point-process model with optimization to compute variation of physiological data (e.g., HRV) with greater computation efficiency and better interpretability of health than statistical models traditionally utilized. For example, in many embodiments, a framework comprises a point-process model with global optimization utilizing gamma distribution of parameter data to fit physiological interval data (e.g., heartbeat intervals) and then compute variation of the physiological parameter data (e.g., HRV). The increased efficiency of point-process models with optimization allows for enhanced retroactive analysis and more efficient real-time analysis than prior statistical models (e.g., Barbieri IG model). Furthermore, point-process models with optimization can utilize a log-link function that yields weights that can elucidate the contribution of a variable on the output. And unlike prior statistical models that needed to constrain weights, the weights output by this model are unconstrained, and thus their effect on the physiological parameter can be readily interpreted based on numerical sign and scale (i.e., further from 0, greater positive or negative effect). These weight outputs can be further utilized in downstream applications, such as, for example, training a machine-learning model to predict a health characteristic using the weights as input into the model. A trained machine-learned model can be utilized detect a health characteristic based on physiological variability (e.g., detection of an effector and/or diagnosis of an underlying medical disorder from captured physiological data). And because the point-process models with optimization efficiently computes physiological variability, detection of a health characteristic can be done in real-time. But notably, detection and/or diagnostics of a health characteristic can also be done after collection of physiological data, which may be useful in diagnostic setting, for example, when trying to assess for a particular health characteristic (e.g., autonomic disorder) from prior data collected.

In several embodiments, a computational framework further comprises state-space model to compute influences on parameter variation that arises from underlying contributions from biological systems (e.g., computing the influence of respiration on heartbeat variance). Covariate data can be collected in addition to the physiological data and assessed. For example, in many embodiments, a computational framework comprises a state-space model with global optimization using a gamma distribution to assess the relationship of covariate data on physiological data. State-space models with convex optimization can efficiently and effectively determine the relationship of a covariate with physiological parameter variation, allowing for rapid and precise temporal detection of effectors on and/or effects of the physiological parameter, such that covariate relationships can be detected real-time and/or precisely diagnosed after collection of data. These models can be applied in a way that allows for monitoring of potential effectors in real time (e.g., mechanical ventilation on heartbeat variation), diagnostic identification of an effector (e.g., identification of an underlying condition causing abnormal HRV) or precise temporal diagnosis of effectors that resulted physiological parameter change (e.g., precise determination of a stress event that caused abnormal physiological parameter values). These models can be applied in a way that allows for monitoring of potential effects of the physiological parameter variation, which can be useful in determining other biological systems that are affected and may need attention.

Several embodiments are directed towards computing heart rate and HRV with high temporal resolution. In many embodiments, a computational framework enhances computation of heartbeat dynamics. In some embodiments, a computational method can receive heartbeat interval data, which can be captured utilizing a sensor. In some embodiments, a point-process model with global optimization is fit to the heartbeat interval data. In some embodiments, the point-process model with optimization comprises utilization of a distribution for a convex solution, such as a gamma distribution. In some embodiments, the model fit to the heartbeat interval data is utilized to compute heart rate and HRV. The HRV can be displayed or otherwise provided to a user or clinician. The HRV can be further utilized in a number of downstream applications.

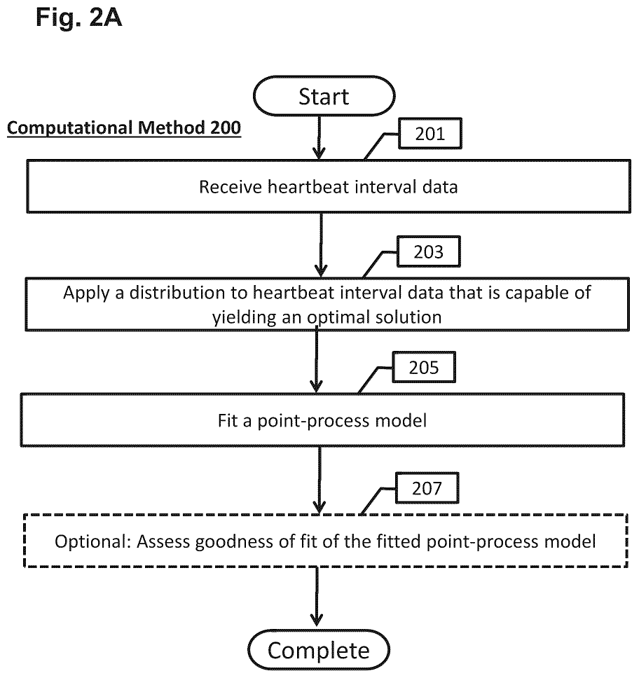

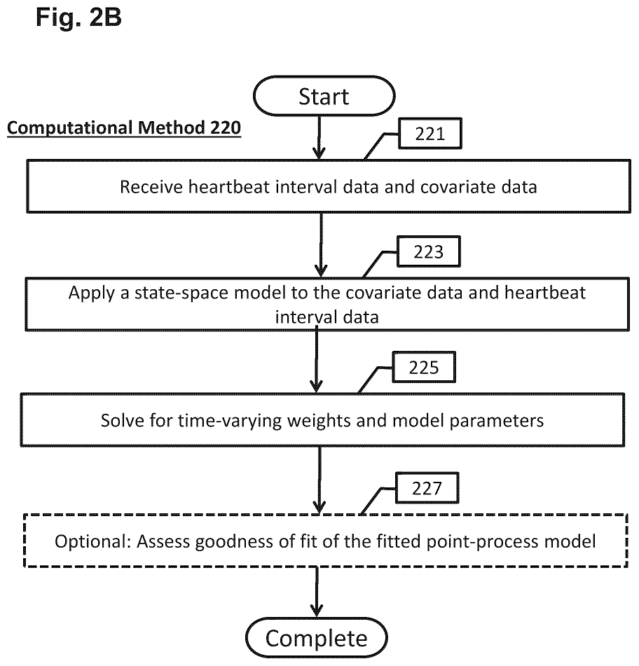

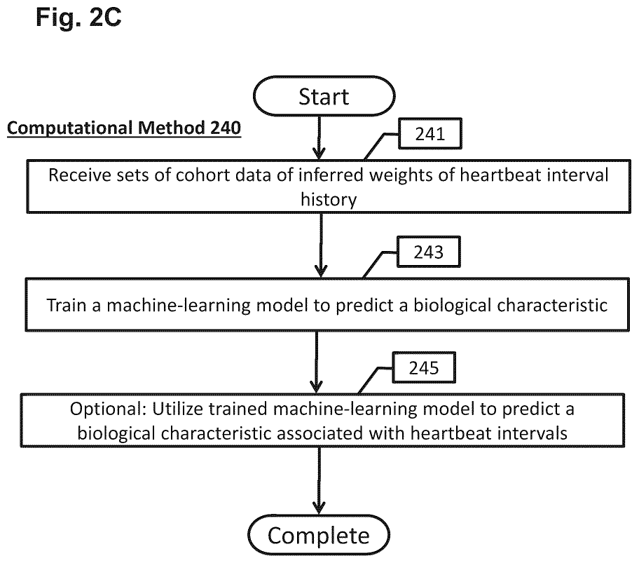

Provided in FIG. 2A is an example of a computational method to compute HRV with high temporal resolution. The computational method can be performed on any computational system with a processor system. In some implementations, the computational method is performed on a health monitoring system, such as an ECG system or a hemodynamic monitoring system. In some implementations, the method is performed retroactively, after heart rate data is sensed. In some implementations, the method is performed in real time, in which computation of HRV is performed as heart rate is sensed, which can be performed with minimal to no delay.

Computational method 200 can begin by receiving (201) heartbeat interval data. The heartbeat interval data can be captured by a sensor and transmitted to a computational processing system for fitting a model. In some implementations, the heartbeat interval data is historical data previously captured. In some implementations, heartbeat interval data is captured by a sensor and received in real time.

Various sensors can be utilized to capture heartbeat interval data. Generally, any sensor capable of capturing heartbeat intervals, or capturing data that can be utilized to derive a heartbeat can be utilized. The sensor can be invasive or noninvasive. In some implementations, a sensor is capable of measuring electrical signals of the heart. An example of a sensor for measuring electrical signals of the heart include (but is not limited to) one or more leads of an electrocardiography (ECG) machine or ECG-like device (such as those incorporated within wearable devices). In some implementations, a sensor is capable of measuring blood pressure. Examples of sensors for measuring blood pressure include (but are not limited to) a blood pressure transducer catheter (e.g., intra-arterial catheters, pulmonary wedge catheters, cardiac catheters, and intravenous catheters), a blood pressure cuff, and an ultrasound transducer. In some implementations, a sensor is capable of measuring blood flow. Examples of sensors for measuring blood flow include (but are not limited to) an ultrasound transducer and magnetic resonance imaging (MRI) scanner. In some implementations, a sensor is capable of measuring blood volume. An example of a sensor for measuring blood volume includes (but is not limited to) a photoplethysmograph (PPG).

Computational method 200 can apply (203) a distribution to the heartbeat interval data that is capable of yielding an optimal solution. In many embodiments, the distribution is one that is a shape that yields a concave or convex function. In some embodiments, the distribution selected is a gamma distribution, which is a two-parameter distribution with a shape parameter and scale parameter.

To obtain a distribution that yields an optimal solution, a random vector can be defined for interbeat intervals between successive heartbeats. The interbeat intervals can be modeled as statistically dependent on recent history. For example, a regression model of with a distribution that yields a concave or convex function can be utilized. Various regression models can be utilized, such as linear regression, logistic regression, Poisson regression, Bayesian regression, or generalized linear model (GLM). In some particular embodiments, the regression model is a GLM and the distribution of data is a gamma distribution. A GLM can comprise a link function for better interpretability of a fitted model.

Computational method 200 can fit (205) a point-process model. Generally, the point-process model has convex optimization to yields a global optimum, as would be achievable based on the distribution of the heartbeat interval data. In some implementations, the point-process model comprises the use of a gamma-distributed GLM with a link function to model the conditional expectation of the current heartbeat interval as an affine combination of the past p heartbeat intervals (where p is the order of the model). In some implementations, the link function is a log-link function. The past heartbeat intervals can be weighted, wherein the weights reflect the past timing of intervals. In some implementations, the weights are inferred as a convex optimization problem. In some implementations, once weights are inferred, the shape parameter of the gamma distribution is inferred, which can be used to infer the scale parameter.

Because of the solution utilizes a convex function, the inferred weights do not need to be nonnegative and are otherwise not constrained, and thus the weights are affine and can be useful for interpretation of the fitted model. A positive weight indicates that the parameter increases heart rate and conversely a negative weight indicates that the parameter decreases heart rate. And the amount of increase or decrease on heart rate is proportional to the magnitude of the weight. As is discussed in greater detail in reference to FIG. 2C, the inferred weights can be utilized to interpret health characteristics associated with the heartbeat interval data via training of a machine-learning model.

Computational method 200 can optionally assess (207) goodness of fit of the point-process model, which can help determine whether the model accurately represents HRV. Any methodology to assess goodness of fit can be utilized. In some implementations, goodness of fit is determined utilizing Kolmogorov-Smirnov (KS) distance. In some implementations, assessment of goodness of fit further comprises the use of time rescaling.

For more on a gamma GLM, see the example provided herein of deriving a gamma GLM. The performance of the derived Gamma GLM was assessed and compared to an inverse Gaussian point-process model. The gamma GLM took less time and computational effort to fit a model than the inverse Gaussian point-process model. With large amount heartbeat interval data, the Gamma GLM further improved fit, as determined by KS distance.

With a point-process model with convex optimization fitted, heart rate and HRV can be computed with the model. The fitted model can yield high temporal resolution of the HRV or any other hemodynamic parameter derived therefrom. Because of the computational efficiency, the model can be fitted and then used to compute HRV as a sensor captures data (or immediately thereafter). In some implementations, the fitted model can be used to compute continuous time estimates of heart rate and HRV, which can be displayed in real time. In some implementations, the model is used to assess an individual's autonomic nervous system in real time, which may be useful in various medical settings. In some implementations, the model may be used to describe neural process or for real-time measurements of neuron firing rates.

While specific implementations of methods to compute heart rate variability are described above, one of ordinary skill in the art can appreciate that various steps of the method can be performed in different orders and that certain steps may be optional according to various implementations. As such, it should be clear that the various steps of the method could be used as appropriate to the requirements of specific applications. Furthermore, any of a variety of methods compute heart rate variability appropriate to the requirements of a given application can be utilized in various implementations.

Provided in FIG. 2B is an example of a computational method to compute covariate relationship with heartbeat dynamics. The computational method can be performed on any computational system with a processor system. In some implementations, the computational method is performed on a health monitoring system, such as an ECG system or a hemodynamic monitoring system. In some implementations, the method is performed retroactively, after heart rate and covariate data is sensed. In some implementations, the method is performed in real time, in which computation of covariate relationship is performed as heart rate and covariates are sensed, which can be performed with minimal to no delay.

Computational method 220 can begin by receiving (221) heartbeat interval data and covariate. The heartbeat interval data can be captured by a sensor and processed as described in computational method 200. Covariate data is to mean any data of biological processes that covary with heartbeat intervals. In some implementations, the heartbeat interval data is historical data previously captured. In some implementations, heartbeat interval data is captured by a sensor and received in real time.

Any covariate data can be captured and assessed. Examples of covariates include several biological characteristics associated with the autonomic nervous system, including (but not limited to) respiration, sleep, stress, posture, inflammation, and drugs. Data collection of these covariates can be performed in any appropriate manner, such as the use of sensors and/or patient history. For nonlimiting examples, respiration can be measured via capnography; sleep and posture can be measured by accelerators; posture can also be assessed by a tilt-table assessment; stress can be measured by patient history and/or blood pressure; inflammation can be measured by blood tests (e.g., C-reactive protein, fibrinogen, white blood cell count, etc.); and drugs can be measured by patient history and/or direct measurement in the blood.

Computational method 220 can apply (223) a state-space model to the covariate data and heartbeat interval data. The state-space model can be utilized in conjunction with a point-process model, such as the one described within computational method 200. Generally, a state-space model is designed to acquire multiple copies of state and use a high dimensionality solution with optimization (e.g., convex or concave solution). In some implementations, a latent process model is utilized, which can help capture the cause and effect of covariates upon HRV. In some implementations, the latent process model is a Gauss-Markov process.

Computational method 220 can solve for time-varying weights and model parameters. To solve for time-varying weights, a high dimensionality solution with optimization such as the Gauss-Markov process is utilized. Due to the high dimensionality, in some implementations, an alternating direction method of multipliers (ADMM) is utilized. In some embodiments, model parameters can be solved as described in computational method 200.

Computational method 220 optionally assess (227) goodness of fit of the state-space model, which can help determine whether the model accurately represents HRV. Any methodology to assess goodness of fit can be utilized. In some implementations, goodness of fit is determined utilizing Kolmogorov-Smirnov (KS) distance. In some implementations, assessment of goodness of fit further comprises the use of time rescaling.

While specific implementations of methods to compute covariate relationship with heartbeat dynamics are described above, one of ordinary skill in the art can appreciate that various steps of the method can be performed in different orders and that certain steps may be optional according to various implementations. As such, it should be clear that the various steps of the method could be used as appropriate to the requirements of specific applications. Furthermore, any of a variety of methods compute covariate relationship with heartbeat dynamics appropriate to the requirements of a given application can be utilized in various implementations.

Provided in FIG. 2C is an example of a computational method to train a machine-learning model to predict a biological characteristic associated with heartbeat intervals. The computational method can be performed on any computational system with a processor system. In some implementations, the computational method is performed on a health monitoring system, such as an ECG system or a hemodynamic monitoring system. Generally, the machine-learning model can be trained to predict a biological characteristic that is detectable from HRV. In some embodiments, the machine-learning model is trained to predict a biological characteristic associated with the autonomic nervous system. Examples of biological characteristics associated with the autonomic nervous system include inflammation, stress, cardiovascular function, metabolic function, liver function, renal function, and nervous system function. In some embodiments, the machine-learning model is trained to predict the risk of a medical disorder that covaries with the autonomic system. Examples of these medical disorders include orthostatic hypotension, postprandial hypotension, multiple system atrophy, pure autonomic failure, afferent baroreflex failure, familial dysautonomia, heart failure, metabolic disorder, diabetes, and cardiovascular disease.

Computational method 240 can begin by receiving (241) sets of cohort data of inferred weights of heartbeat interval history. The inferred weights of heartbeat interval history can be generated using a function with global optimization, such as the point-process model described in FIG. 2A or the state-space model described in FIG. 2B.

At least two sets of cohort data of inferred weights of heartbeat interval history should be received. Generally, cohorts are selected based on differing biological characteristics. For example, a common set of cohorts can be a cohort having a medical disorder and a cohort not having the medical disorder (generally referred to as a healthy cohort). But any sets differing biological characteristics can be selected, including sets that are nonbinary (e.g., nondiabetic, prediabetic, and diabetic). The biological characteristics can be a difference in analyte measurements, a difference in patient histories, presence or absence of medical disorder, or any other differing biological characteristics. In many embodiments, the biological characteristic is associated with the autonomic nervous system.

Computational method 240 can train (243) a machine-learning model to predict a biological characteristic associated with the sets of cohort data. Any machine-learning model can be utilized, such as a regressor or a classifier. A regressor may be preferred when predicting a likelihood of biological characteristic. A classifier may be preferred when predicting the presence of a biological characteristic.

To train a machine learning model, the inferred weights of heartbeat interval history from individuals of each cohort is utilized, labelling the inferred weight data with the biological characteristic associated with the cohort phenotype. Upon training, assessment of the accuracy and sensitivity of the model to predict the biological characteristic can be determined.

Computational method 240 can optionally utilize (245) the trained machine-learning model to predict a biological characteristic associated with heartbeat intervals. Heartbeat interval data from a patient can be entered into the model, yielding a prediction whether the patient has a likelihood for or classifies with biological characteristic.

While specific implementations of methods to train a machine learning model to predict a biological characteristic associated with heartbeat interval history are described above, one of ordinary skill in the art can appreciate that various steps of the method can be performed in different orders and that certain steps may be optional according to various implementations. As such, it should be clear that the various steps of the method could be used as appropriate to the requirements of specific applications. Furthermore, any of a variety of methods compute a biological characteristic associated with heartbeat interval history appropriate to the requirements of a given application can be utilized in various implementations.

### Health Monitoring System

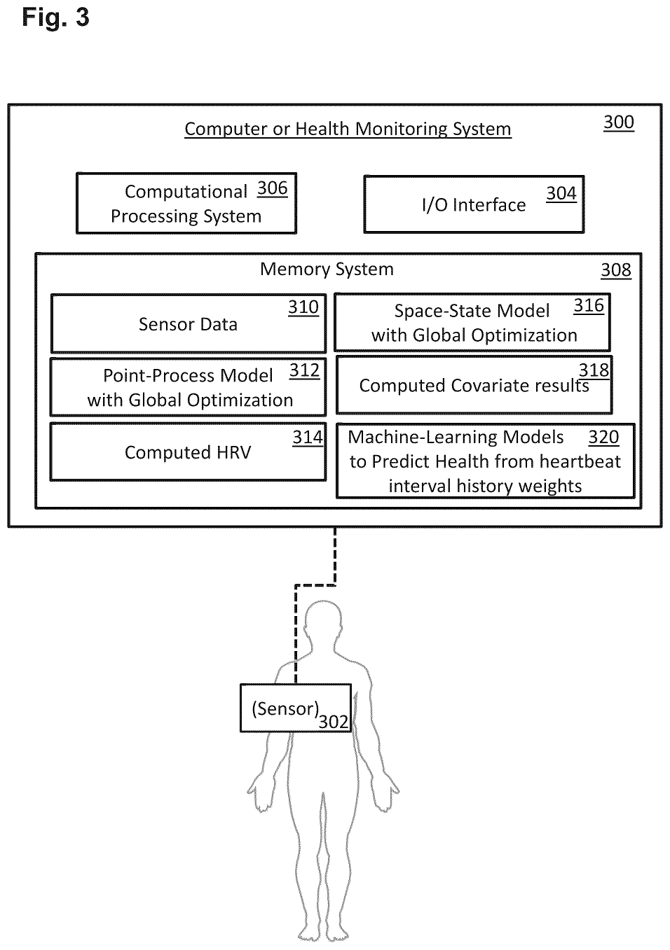

The systems and methods of the current disclosure can be utilized within a computer or health monitoring system. Generally, a computer or health monitoring system comprises computational processing system, and one or more applications to perform the various computational methods described herein. A health monitoring system can further comprise a sensor. Provided in FIG. 3 is an example of a computer or health monitoring system 300. In some implementations, the health monitoring system can be a wearable device (e.g., smart watch) a clinical monitoring system (e.g., hemodynamic monitoring system; e.g., ECG device), or any other health monitoring system having a sensor and a computational processing system for performing applications of the computational methods described herein.

Health monitoring system can comprise a sensor 302 for capturing data (e.g., heartbeat interval data or other sensor data that can be used to derive heartbeat interval data). Sensor 302 can be in connection with computational processing system 306 such that the captured sensor data can be transmitter thereto. Sensor 302 can be an invasive sensor or a noninvasive sensor. Various sensors can be utilized, including, but not limited to: one or more leads of an ECG, a blood pressure transducer catheter, a blood pressure cuff, an ultrasound transducer, MRI scanner, or a PPG. In some implementations, the sensor is housed in a wearable device, such as a lead of an ECG or a PPG.

Computer or health monitoring system 300 can comprise a computational processing system 306 for running one or more applications and/or models and an I/O interface 304 for input and output of data, such as data communicated between sensor 302, a user interface, and/or a display screen. Computer or health monitoring system 300 can include a memory system 308 that can store data, applications, and/or models.

Computational processing system 306 typically utilizes a processing system including one or more of a CPU, GPU and/or neural processing engine. As described herein, sensor data can be received and transformed into HRV with high temporal resolution in real time using the computational processing system.

Computational processing system 306 can be housed within a computer, tablet, smart phone, wearable device or a health monitor in digital connection with various components, inclusive of a sensor. In some implementations, the computational processing system 306 can be housed separately from a wearable device or the patient monitor (e.g., within computer, tablet, or smart phone), but receives the acquired sensor data (e.g., wired, WiFi, cellular, Bluetooth, etc).

In the illustrated example, memory system 308 is capable of storing various data, applications, and models. It is to be understood that the listed data, applications and models are a representative sample of what can be stored in memory and that various memory systems may store some or all of the various data, applications, and models listed. Further, any combination of data, applications, and models can be stored, and in some implementations, various data, applications, and/or models are stored temporarily.

In some implementations, the memory system 306 can store acquired sensor data 310, which can be obtained from sensor 302. A point-process model with global optimization 312 and a state-space model with global optimization 316 can be stored in memory system 308 utilize sensor data 310 to fit a model the sensor data and/or compute heart rate and heart rate variation. The computed HRV 314 and covariate results 318 (or any other computed parameter, such as heartbeat interval history weights) can be stored in memory system 308. Further, any data or resulting data, such as the prior and/or real-time HRV 314 and covariate results 318 (or any other computed parameter, such as heartbeat interval history weights) can be displayed on a screen via the I/O interface 304.

In some implementations, the memory system 306 can comprise one or more machine-learning models to predict health from heartbeat interval history weights 320. The machine learning model can be utilized to predict a biological characteristic associated with the heartbeat intervals. The results of the machine-learning models can be be displayed on a screen via the I/O interface 304.

While a specific health monitoring system configuration is described above with reference to FIG. 3, it should be readily appreciated that various health monitoring systems utilized in the provision of HRV assessment can be implemented in any of a variety of configurations. Accordingly, the various systems and methods described herein should be understood as not to be limited to any specific computer or health monitoring system, but instead can be implemented using any variety of computers or health monitoring systems capable of performing the various computational methods described herein.

### EXAMPLES AND DATA RESULTS

The systems and methods of the current disclosure will be better understood with the several examples and data results provided. Validation results are also provided. The examples and data results provide evidence that the described methods for determining hemodynamic parameter variability are able to effectively assess heartbeat dynamics and the effects of covariates. The examples and data results refer to a history-dependent Gamma generalized linear model (GLM) that provided assessments of ECG-derived data by determining a convex solution to yield an optimized fit model. The current models developed were benchmarked against the inverse Gaussian model of Barbieri et al., (Am J Physiol Heart Circ Physiol. 2005, 288 (1): H424-H435, the disclosure of which is hereby incorporated by reference).

### Example 1: A Convex Formulation of Point Process Heartbeat Dynamics Using a Gamma Generalized Model

Heartbeat dynamics (such as HRV) have long been a staple of experiments and analyses of the autonomic nervous system, including how sleep, emotions, pain, and stress are regulated in health and disease. The autonomic nervous system is the branch of the nervous system that governs all unconscious reflexes and responses and interfaces with other body systems, including the cardiovascular system. It is possible to infer autonomic nervous system activity from heartbeat dynamics because the beat-to-beat variation in individual heartbeats contains information about the sympathetic (“fight-or-flight”) and parasympathetic (“rest-and-digest”) inputs from the brain on a second to second basis.

However, most approaches to extract this information do not achieve this level of temporal resolution, largely because they rely on temporal and spectral windowing and smoothing approaches. Approaches that do achieve true instantaneous temporal resolution do so because they take advantage of the point process nature of heartbeat generation. Heartbeats are generated via cardiac action potentials, which are discrete events that occur in continuous time.

One example of an approach that successfully captures and models this point process phenomenon is that developed by Barbieri et al., (Am J Physiol Heart Circ Physiol. 2005, 288 (1): H424-H435, the disclosure of which is hereby incorporated by reference). In this approach, the membrane potential of cardiac pacemaker cells is modeled as a Gaussian random walk with linear drift, and in doing so, an inverse Gaussian model is hypothesized for an interval between successive heartbeats such as the RR interval. However, one potential challenge of this method from the standpoint of implementation is that the maximum likelihood estimation problem is non-convex, which means that a local optimum of a model fitting procedure is not necessarily a global optimum. Judicious choice of initial conditions for model fitting procedures is required, which makes the procedure more computationally expensive.

In this example, we provide a history-dependent Gamma generalized linear model (GLM) framework for heartbeat dynamics that retains all of the benefits of the Barbieri et al. framework while also rendering the maximum likelihood estimation problem convex. The ease of implementation and less computation requirements will lead to greater usage by non-experts and a real-time monitoring system.

### Methods

### A History-Dependent Gamma GLM Point-Process Model

We can model a continuous non-negative random variable Y with a Gamma distribution with shape and rate parameters (α,β) as:

$${f}_{Y} ( y ; α , β ) = \frac{{y}^{α - 1} {e}^{- β y} {β}^{α}}{Γ ( α )} , \\ y > 0 , α , β > 0 \qquad ( Equation 1 - 1 )$$

where Γ(·) is the Gamma function.

While past point process heartbeat dynamics work utilizes a history-dependent inverse-Gaussian (IG) distribution, since an IG models the first-passage times of a random walk. We here choose a Gamma distribution as a natural analog to recast physiology that an inter-beat interval is statistically dependent on recent past sympathetic and parasympathetic inputs to the heart.

Define the random vector, Y≙(Y₁, Y₂, . . . , Yₙ), as the interbeat intervals (in seconds) between successive heartbeats, where any Yⱼ∈ [symbol] ₊ is nonnegative. As such, we model each RR interval Yⱼ as statistically dependent on its recent history [symbol] ⱼ=(Yⱼ₋₁, . . . , Yⱼ₋ₚ). A natural way to do this is by constructing a Gamma GLM as follows:

$$log 𝔼 [ {Y}_{j} ] \vert {ℋ}_{j} = {h}_{j} ] = {w}_{0} + {\sum }_{k = 1}^{p} {w}_{k} {y}_{j - k} ≡ L ( w ; {h}_{j} ) , \qquad ( Equation 1 - 2 )$$

where w₀, w₁, . . . , wₚ are a fixed set of weights that connect the expectation of Yⱼ to its history. The log link is used to map [symbol] [Yⱼ| [symbol] ⱼ=hⱼ]∈ [symbol] ₊ onto [symbol] , since w₀, w₁, . . . , wₚ can take on any real value. This then describes a point process model where the distribution of any RR interval conditioned upon its past is described by a Gamma distribution.

### Model Fitting

For the Gamma density with parameters (α, β), we have from Equation 1-1 that

$$- log {f}_{Y} ( y ; α , β ) = log Γ ( α ) - ( α - 1 ) log y + β y - α log β . \qquad ( Equation 1 - 3 )$$

Given the expectation of a Gamma random variable is

$$𝔼 [ Y ] = \frac{α}{β} ,$$

we have that for a Gamma GLM with log link function:

$$log ( \frac{α}{β} ) = L ( w ; {h}_{j} ) . \qquad ( Equation 1 - 4 )$$

We note from the above equation that the parameters of the model can now be described in terms of a and w.

We first consider the case where there is one RR interval of duration y and history h. Then the negative log likelihood described in terms of shape parameter a and weights w is given by

$$NLL ( α , w ) = log Γ ( α ) - ( α - 1 ) log y + α y {e}^{- L ( w ; h )} + α L ( w ; h ) - α log α \qquad ( Equation 1 - 5 )$$

Then for any a>0, the maximum likelihood (ML) estimator for w is given by

$$\begin{matrix}\hat{w} = {arg min}_{w} NLL ( α , w ) \\ = {arg min}_{w} α ( y {e}^{- L ( w ; h )} + L ( w ; h ) ) \\ = {arg min}_{w} y {e}^{- L ( w ; h )} + L ( w ; h )\end{matrix} \qquad \begin{matrix}\begin{matrix}( Equation 1 - 6 ) \\ \end{matrix} \\ ( Equation 1 - 7 )\end{matrix}$$

where Equation 1-6 follows from elimination of terms in the sum that do not depend on w and Equation 1-7 follows from the fact that a>0. If we now have J heartbeat intervals, the ML estimator for w is given by

$$\hat{w} = arg {min}_{w} {\sum }_{j = 1}^{J} {y}_{j} {e}^{- L ( w ; {h}_{j} )} + L ( w ; {h}_{j} ) \qquad ( Equation 1 - 8 )$$

Note that this minimization problem is convex in w and does not force weights to be nonnegative. It thus overcomes two limitations of the IG approach of Barbieri et al., (Am J Physiol Heart Circ Physiol. 2005, 288 (1): H424-H435, the disclosure of which is hereby incorporated by reference).

After finding the ML estimate for w, we can find the ML estimate for a as follows. Define Lⱼ≙L(ŵ; hⱼ). Taking into consideration all terms from the NLL that involve a, we have from that:

$$\hat{α} = arg {min}_{α} J log Γ ( α ) - α J log α - {\sum }_{j = 1}^{J} ( α - 1 ) log {y}_{j} + α {y}_{j} {e}^{- {L}_{j}} + α {L}_{j} . \qquad ( Equation 1 - 9 )$$

Since we have both â and ŵ, we can directly compute β\hat{ } through Equation 1-4.

### Goodness of Fit

To assess goodness of fit of our model, we follow the methodology proposed by Barbieri et al., through the time-rescaling theorem. First, define the times at which R-wave events occur, 0<u₁<u₂< . . . <uₖ<T, where Tis the total time of recording. We can express any uₖ as the sum of all interbeat intervals leading up to uₖ,

$${u}_{k} = {\sum }_{i = 1}^{k} {y}_{i} . \qquad ( Equation 1 - 10 )$$

The conditional intensity of the point process can be described as:

$$λ ( t \vert {ℋ}_{t} ; \hat{θ} ) = {f ( t \vert {ℋ}_{t} ; \hat{θ} ) [ 1 - {\int }_{{u}_{{n}_{t}}}^{t} f ( v \vert {ℋ}_{v} ; \hat{θ} ) dv ]}^{- 1} , \qquad ( Equation 1 - 11 )$$

where θ\hat{ }=(â,ŵ), the parameters fit by the model, uₙₜ<t, is the time at which the most recent previous R-wave event occurred, and f(t| [symbol] ₜ; θ\hat{ }) is the Gamma GLM density with history t evaluated at time t uₙₜ, which by definition of uₙₜ is non-negative.

We can then compute the time-rescaled interbeat intervals by

$${τ}_{k} = {\int }_{{u}_{k - 1}}^{{u}_{k}} λ ( t \vert {ℋ}_{t} ; \hat{θ} ) dt . \qquad ( Equation 1 - 12 )$$

The time-rescaling theorem states that for an arbitrary point process, the time-rescaled inter-event intervals (Tₖ:k≥1) as defined above are independent exponentially distributed random variable with unit rate. We can assess the goodness of fit by the transformation Zₖ=Fₑₓₚ(Tₖ), where Fₑₓₚ(s)=1−e^{-s} is the cumulative distribution function (CDF) of a unit rate exponential random variable. Since it is well-known that a inputting a random variable into its own CDF produces a uniform random variable on [0, 1], the (Zₖ:k≥1) should be uniformly distributed on [0, 1] if the data did indeed come from the model. We can compare the (Zₖ:k≥1) to the uniform random variable on [0, 1] by means of a Kolmogorov-Smirnov (KS) plot. We then calculate the KS distance between the empirical CDF of (Zₖ:k=1), . . . , n) and compare to the theoretical CDF of the uniform distribution F_{U}(·) as:

$$D = {sup}_{u} \vert {F}_{U} ( u ) - {F}_{emp} ( u ) \vert , \qquad ( Equation 1 - 13 )$$

where F_{U}(·) is the CDF of a uniform random variable on [0,1] and Fₑₘₚ(·) is the empirical CDF of the Zₖ. This metric measures the level of dissimilarity between the distributions.

To assess performance of the proposed algorithm compared to the existing algorithm, we collected a healthy human dataset of over 7000 RR intervals from and fit both algorithms to the data. The data were collected from a human subject wearing an ambulatory electrophysiology monitoring device as described in Gharibans et al., (Sci Rep. 2018 Mar. 22; 8 (1): 5019, the disclosure of which is hereby incorporated by reference). Both the amount of time it took to fit the algorithm and the KS distance were calculated to assess computational time and model fit. Both lower runtime and lower KS distance indicate a better performing algorithm. Since the initial portion of a recording can tend to have unreliable measurements, we chose a window of 120 seconds at the beginning to exclude from the analysis. We chose a model order, p=6, as in Barbieri et al., by the Akaike Information Criterion (AIC) for the IG model, and set it to be the same for the Gamma model for comparison. We then and ran a sweep to fit models for J [100, 7250]. Specifically we ran the model for J=100 and increased 100 intervals up to J=1000, then every 250 intervals up to J=7250.

### Results

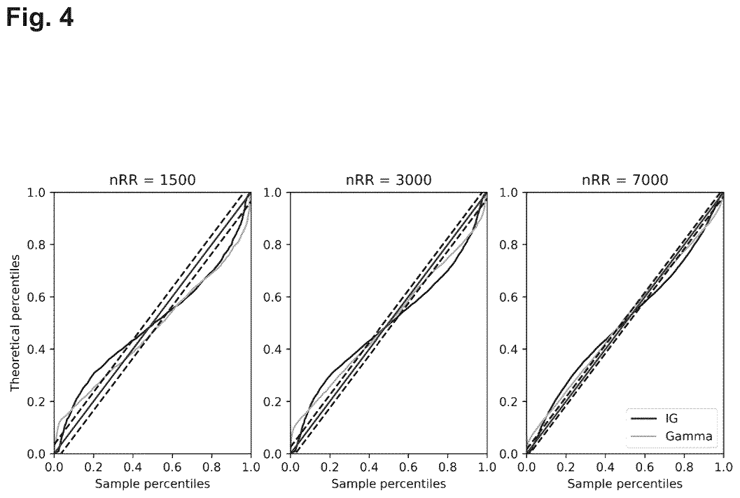

Overlaid KS plots of both models for the number of RR intervals fit, J, are shown in FIG. 4. We choose J=1500, 3000, and 7000 to be representative of the difference in model fit. The y-axis represents the theoretical percentiles of F_{U}(·), a uniform distribution on [0,1], while the x-axis represents the percentiles of Fₑₘₚ(Tₖ) from the actual sample data. The central diagonal line indicates perfect fit and the diagonal dashed lines represent the 95% confidence interval for the data coming from the model. For J=1500, we can see that the IG model as a very slight advantage in model. However, this advantage disappears as J increases. At J=7000, we can see that the Gamma model outperforms the IG model in goodness of fit, indicating that the Gamma model performs better for larger datasets. While neither model lies within the confidence intervals in the KS plot (indicating that we don't have a perfect model fit), we note that both model fits are very close to the central diagonal line. This indicates to us that both point process models have good fit.

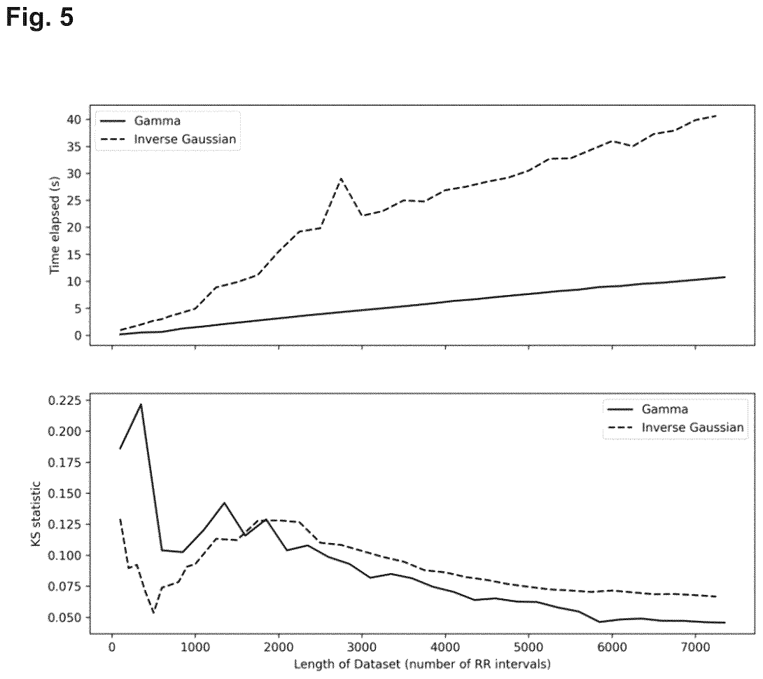

In FIG. 5, the runtime and KS distance for each model are plotted against the number of RR intervals fit. Lower values of KS distance indicate better model fit. In the figure we can see that all sets of RR intervals fit, the Gamma model had a much faster runtime (4× on average). For J>1750, the Gamma model also has lower KS distances than the IG model, which indicated for larger J, the Gamma model has better fit than the IG model.

### CONCLUSIONS

In this example, we have shown that our history-dependent Gamma point process model outperforms the original IG point process model in speed and model fit for when data input is increased. We measured computational speed using runtime of the algorithm, and model fit through the KS distance. Both FIG. 4 and FIG. 5 validate our model with low runtimes and KS distances. While our original motivation for choosing a Gamma GLM to fit the model was in its convexity, for speed and ease of use, we found that it provides a better fit in larger datasets.

We also must note that since the Gamma distribution was chosen for its convenient computational properties, but it may not follow the physiology of the cardiac system as closely as the IG model. This is because the original formulation of the IG model accounts for a Gaussian random walk model of the cardiac membrane potential, which has an IG density describing the interbeat intervals. But, the Gamma distribution model was the better model fit, which may be because the Gamma distribution is much more flexible in shape than the IG distribution. This means it can better handle noise or some other unknown physiological process represented in the data, in addition to heartbeat dynamics.

This approach can be expanded to calculate continuous time estimates of heart rate (HR) and heart rate variability (HRV). In particular, state-space methods can be used to estimate dynamically changing weights, which will give us an estimate of HR and HRV. For example, using a Kalman Filter-like approach would allow for real-time applications of the heartbeat point process model. This would be particularly advantageous to understand the function of the autonomic nervous in the operating room during surgery. A key innovation is the computational time of the model, which is advantageous for real-time applications that would benefit due to faster compute times and a simpler algorithm.

In addition, in recent literature, a Gamma renewal process has shown to arise from a diffusion leaky-integrate-and-fire process (P. Lansky, et al., Biol Cybern. 2016 Jun.; 110 (2-3): 193-200, the disclosure of which is hereby incorporated by reference). This allows use of our model to better physiologically describe neural processes and be used for real-time measurements of neural firing rates.

In conclusion, we have developed a convex formulation of the point process heartbeat dynamics model that has lower runtimes and good model fit. The model offers an alternative approach to existing methods for ease-of-use for non-experts in signal processing to better understand the time-varying dynamics of the cardiovascular system.

### Example 2: An Efficient Framework for Solving a Convex, State-Space Heartbeat Dynamics Model

Analyzing dynamics between successive heartbeats, such as RR intervals, has been used to assess the health of a person's autonomic nervous system (ANS). For example, heart rate recovery within a minute after exposure to physical stress or psychological stress has been used to measure vagal activity and was found to be a predictor of overall mortality (see, e.g., C. R. Cole, et al., N Engl J Med. 1999 Oct. 28; 341 (18): 1351-7 and E. S. Mezzacappa, et al., Psychosom Med. 2001 July-Aug; 63 (4): 650-7; the disclosures of which are hereby incorporated by reference). Heartbeat dynamics also aid in the understanding and diagnoses of conditions such as epilepsy, orthostatic hypotension, and diabetic autonomic neuropathy (See, e.g., T. Tomson, et al., Epilepsy Res. 1998 March; 30 (1): 77-83; J. B. Lanier, et al., Am Fam Physician. 2011 Sep. 1; 84 (5): 527-36; and A. I Vinik, et al., Diabetes Care. 2003 May; 26 (5): 1553-79; the disclosures of which are hereby incorporated by reference).

Traditional methods for analyzing heartbeat dynamics utilize a spectral analysis via a windowing technique (A. Malliani, et al., Circulation. 1991 August; 84 (2): 482-92; the disclosure of which is hereby incorporated by reference). Since then, other models for heartbeat dynamics have been developed (see, R. Bailon, et al., IEEE Trans Biomed Eng. 2011 March; 58 (3): 642-52; and F. E. Rosas, et al., Comput Biol Med. 2024 Mar.; 170:107857; the disclosures of which are hereby incorporated by reference). The inverse Gaussian (IG) point process model first introduced by R. Barbieri, et al., (Am J Physiol Heart Circ Physiol. 2005, 288 (1): H424-H435, the disclosure of which is hereby incorporated by reference). The IG model was later incorporated into a state-space model (SSM) (R. Barieri and E. N. Brown, EEE Trans Biomed Eng. 2006 January; 53 (1): 4-12, the disclosure of which is hereby incorporated by reference). SSMs have been described as more capable to model implicit stimuli and represent nonstationarity in systems (A. C. Smith and E. N. Brown, Neural Comput. 2003 May; 15 (5): 965-91; the disclosure of which is hereby incorporated by reference).

In addition, SSMs enable statistically rigorous methods for assessing goodness of fit via the time-rescaling theorem. However, both the static and dynamic versions of the inverse Gaussian model involve nonconvex model-fitting procedures.

To address the noncovexity issue, a new formulation for heartbeat dynamics using a gamma generalized linear model (GLM) with a log-link function was recently introduced for the static setting (see Example 1, described herein). Though the gamma distribution loses some physiological interpretation, it characterizes a similar class of distributions as the inverse Gaussian (i.e., both have nonnegative support and a pair of parameters). With these changes, the model results in a convex maximum likelihood estimation (MLE) problem. However, it does not capture time-varying dynamics in the statistics.

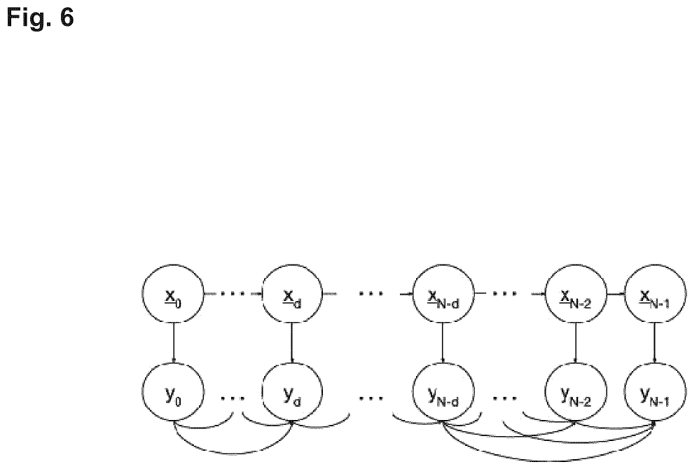

Here we consider the dynamic setting with an SSM using a gamma GLM for the measurement model (FIG. 6). We leverage the log-concavity of the measurement model to develop an alternating direction method of multipliers (ADMM) framework to efficiently compute the maximum a priori (MAP) estimate of the time-varying latent process (G. Schamberg, et al., IEEE Transactions on Signal Processing, vol. 66, no. 12, pp. 3140-3154, 2018; the disclosure of which is hereby incorporated by reference). We test our procedure on electrocardiogram (ECG) recordings from tilt table data and demonstrate goodness of fit. We show the estimated mean heart rate, a measure of heartbeat dynamics, is responsive to postural changes. In particular, when compared with the commonly used local averaging method, our procedure more clearly captures heart rate decelerations in response to different tilt speeds.

### Methods

In this example, we denote a column vector as v and a matrix as x. We denote a random variable X with capitalization and a specific realization x with lower case.

Consider N interbeat intervals as the random vector Y=[Y₀, . . . , Y_{N-1}]^{T}. We assume there is a latent Gauss-Markov process (GMP) X=[X₀, . . . , X_{N-1}] given by

$$\begin{matrix}{\underline{X}}_{n + 1} = {\underline{X}}_{n} + {\underline{V}}_{n} & ∀ n \geq 1 ,\end{matrix} \qquad ( Equation 2 - 1 )$$

where each Xₙ∈ [symbol] ^{d+1} for a model order d we describe below, X₀˜N (μ₀, Σ₀), and each Vₙ˜N (0, Σᵥ). This model is a natural way to capture time-varying changes in the underlying distribution of interbeat intervals. We describe f xₙ|xₙ₋₁(xₙ|xₙ₋₁) succinctly as pₙ(xₙ|xₙ₋₁) and note that for any u, −log pₙ(v|u) is convex in v.

We consider a measurement model that captures the history-dependent properties of heartbeats, i.e. for each RR interval, its statistics are dependent on the recent RR intervals before it. Formally, the d-th order model f Yₙ|xₙ, Y_{0:n-1} (·|xₙ, y_{0:n-1}) is only a function of the current state and the previous d RR intervals:

$${f}_{{Y}_{n} \vert {\underline{X}}_{n} , {\underline{Y}}_{0 : n - 1}} ( {y}_{n} \vert {\underline{x}}_{n} , {\underline{y}}_{0 : n - 1} ) ≡ {q}_{n} ( {y}_{n} \vert {\underline{x}}_{n} , {\underline{h}}_{n} ) ,$$

where hₙ=[1, yₙ₋₁, . . . , y_{n-d}]^{T}. Gamma distributions are similar to RR interval distributions in that they have positive support, are unimodal, and are right-tailed. Therefore, we let the above conditional distribution be a gamma distribution with parameters α and βₙ, which we specify with a GLM to encode the history-dependence:

$$\begin{matrix}log \frac{α}{{β}_{n}} = log 𝔼 [ {Y}_{n} \vert {\underline{X}}_{n} = {\underline{x}}_{n} , {\underline{Y}}_{n - d : n - 1} = {\underline{y}}_{n - d : n - 1} ] \\ = {\underline{h}}_{n}^{T} {\underline{x}}_{n}\end{matrix} . \qquad ( Equation 2 - 2 )$$

Note that for any fixed α, βₙ depends on the previous d RR intervals through a log-link function of the mean. We have that −log qₙ(yₙ|xₙ,hₙ) is convex in xₙ, given by

$$- log {q}_{n} ( {y}_{n} \vert {\underline{x}}_{n} , {\underline{h}}_{n} ) = log Γ ( α ) - α log α - ( α - 1 ) log {y}_{n} + α {y}_{n} {e}^{- {\underline{h}}_{n}^{T} {\underline{x}}_{n}} + α {\underline{h}}_{n}^{T} {\underline{x}}_{n} . \qquad ( Equation 2 - 3 )$$

This fact, along with how −log pₙ(xₙ|u) is convex in xₙ, implies log-concavity of the posterior distribution f X|Y(·|y). Another attractive property of the gamma GLM in Equation 2-3 with the latent GMP in Equation 2-1 is that it is self-consistent. Specifically, each RR interval Yₙ is nonnegative, and we link its conditional expectation to an affine transformation of the latent GMP (which can be positive or negative) via the log-link function in Equation 2-2. In contrast, the inverse Gaussian model in of Barbieri et al. equates the conditional expectation of the positive RR intervals to an affine transformation of the (possibly negative) GMP.

Our goal is to solve for the time-varying weights X∈ [symbol] ^{(d+1)×N} and the parameters α, μ₀, Σ₀, and Σ₀.

### Solving for the Time-Varying, Latent Weights

Assuming all other parameters are known, the MAP estimate of the weights is given by:

$$\begin{matrix}{\hat{x}}_{MAP} = {arg min}_{x ∈ {ℝ}^{( d + 1 ) \times N}} - log {f}_{X \vert \underline{Y}} ( x \vert \underline{y} ) \\ = {arg min}_{x ∈ {ℝ}^{( d + 1 ) \times N}} - log {p}_{0} ( {\underline{x}}_{0} ) - {\sum }_{n = 1}^{N - 1} log {p}_{n} ( {\underline{x}}_{n} \vert {\underline{x}}_{n - 1} ) - \\ {\sum }_{n = 0}^{N - 1} log {q}_{n} ( {y}_{n} \vert {\underline{x}}_{n} , {\underline{h}}_{n} )\end{matrix} . \qquad ( Equation 2 - 4 )$$

Because X is high-dimensional, directly solving this model-even though it is convex-becomes very inefficient when the number of samples N is large. Instead, we use ADMM and introduce auxiliary variables w and z to the equivalent optimization problem based on the framework:

$$( \hat{x} , \hat{w} , \hat{z} ) = {arg min}_{x , w , z} - log {p}_{0} ( {\underline{w}}_{0} ) - {\sum }_{n = 1}^{N - 1} log {p}_{n} ( {\underline{w}}_{n} ) - {\sum }_{n = 0}^{N - 1} log {q}_{n} ( {y}_{n} \vert {\underline{x}}_{n} , {\underline{h}}_{n} ) \\ \begin{matrix}s . t . & {\underline{x}}_{n} = {\underline{z}}_{n} & ∀ n ∈ \{ 0 , … , N - 1 \} \\ & {\underline{w}}_{0} = {\underline{z}}_{0} & \\ & {\underline{w}}_{n} = {\underline{z}}_{n} - {\underline{z}}_{n - 1} & ∀ n ∈ \{ 1 , … , N - 1 \}\end{matrix} .$$

This formulation can be solved iteratively by ADMM where each xₙ update can be solved in parallel, w updates are linear, and z updates are the solutions to Kalman smoothers. This makes it more efficient to solve, and log-concavity of the likelihoods and priors guarantee that ADMM will result in the MAP estimate. In addition, the updates have a statistical interpretation: x considers constraints imposed by the measurement model, w considers constraints imposed by the GMP prior, and z seeks consensus between the two.

### Solving for the Model Parameters

The framework previously described assumes that the model parameters θ=(α, μ₀, Σ₀, Σ₀) are known. In this example, we estimate these parameters using the maximum likelihood estimates (MLE) from the static heartbeat dynamics model as described in Example 1. μ₀ and a are set as \hat{ }x_{MLE} and \ddot{ }a_{MLE}. Σ₀ and Σᵥ are computed by taking a sliding window of 100 intervals and computing \hat{ }x_{MLE} of each shift. Zo is the empirical covariance of these estimates, while Σᵥ is the empirical covariance of the differences of these estimates.

### Validation

We assess model goodness of fit using the time-rescaling theorem (E. N. Brown, et al., Neural Computation, vol. 14, no. 2, pp. 325-346, 2002, the disclosure of which is hereby incorporated by reference). Let 0<u₀<u₁< . . . <u_{N-1}<T be the times of the heartbeats, where Tis the total time of recording. Let qₙ(yₙ|xₙ; θ) be as defined in Equation 2-3 where θ indicates the model parameters. For t∈[uₙ, uₙ₊₁), the conditional intensity λₜ of the heartbeat point process is given by:

$${λ}_{t} = {{q}_{n} ( t \vert {\underline{X}}_{n} , {\underline{h}}_{n} ; θ ) [ 1 - {\int }_{{u}_{n}}^{t} {q}_{n} ( s \vert {\underline{X}}_{n} , {\underline{h}}_{n} ; θ ) ds ]}^{- 1}$$

We define \hat{ }λₜ in terms of the above equation, but with respect to \hat{ }x_{MAP} and the estimated model parameters \hat{ }θ inside the function qₙ. We rescale the RR intervals using

$${τ}_{n} = {\int }_{{u}_{n}}^{{u}_{n} + 1} {λ}_{t} dt , \qquad ( Equation 2 - 5 )$$

and we define T\hat{ }ₙ analogously with respect to λ\hat{ }ₜ.

According to the time-rescaling theorem, (Tₙ, n≥0) are i.i.d. unit-rate, exponential random variables. Therefore, we compute T\hat{ }ₙ with respect to (T\hat{ }ₙ, n≥0) and evaluate goodness of fit by computing \hat{ }Zₙ=Fₑₓₚ(T\hat{ }ₙ), where Fₑₓₚ(s)=1−e^{-s} is the cumulative distribution function of a unit-rate, exponential random variable. If our model is reasonable, then (T\hat{ }ₙ, n≥0) should be approximately i.i.d. unit-rate exponentials, implying that (\hat{ }Zₙ, n≥0) should be approximately i.i.d. Unif ([0, 1]) random variables. We can evaluate this with Kolmogorov-Smirnov (KS) plots and 95% confidence intervals.

The tilt table test is a commonly used clinical test to probe the ANS through postural changes. We test our procedure against publicly available tilt table data from 10 healthy subjects on PhysioNet (T. Heldt, et al., Computers in Cardiology, 2003. IEEE, 2003, pp. 263-266; A. L. Goldberger, et al., Circulation. 2000 Jun. 13; 101 (23): E215-20; the disclosures of which are hereby incorporated by reference). The gender distribution in this dataset is evenly split with mean age reported as 28.7 1.2 years. We consider a subsection of each subject's recordings to specifically compare their heartbeat dynamics during a slow tilt from horizontal to 75° and back versus a rapid tilt along the same trajectory.

We choose model order d=4, which worked the best for most subjects. ADMM was run with p=100 until it reached convergence determined by satisfying the Cauchy criterion for all primal and dual variables. For 9 subjects, this occurred in less than 1000 iterations. In the remaining subject, the ECG recording had artifacts during a significant portion of the data, so we omit them from our analyses.

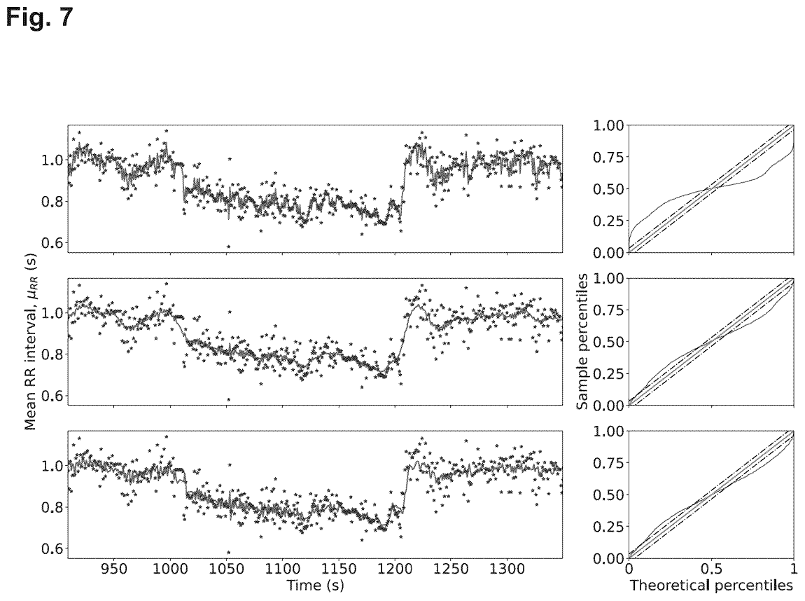

To show the progression of ADMM, using the bx output at each iteration, we compute an estimate of the mean RR interval URR using Equation 2-2. The results shown in FIG. 7 demonstrate how the estimates change as the procedure progresses on subject 12726. In earlier iterations, URR overfits to Y and is reflected by the sharp jump in the KS plot. This is because the x updates are based on information from the measurement model likelihoods. As the iteration number increases, x aims to reach consensus with w, whose updates only contain latent GMP information. This “smooths” the μ_{RR} estimates, resulting in a KS plot approaching the 95% confidence intervals.

We examine the heart rate time series derived from bxMAP, which is an inverse gamma random variable with mean

$${μ}_{HR} [ n ] = \frac{{β}_{n}^{†}}{\hat{α} - 1} = \frac{c \hat{α} {e}^{- {\underline{h}}_{n}^{T} {\underline{x}}_{n}}}{\hat{α} - 1} , \qquad ( Equation 2 - 6 )$$

where c=60 s/min for unit conversion.

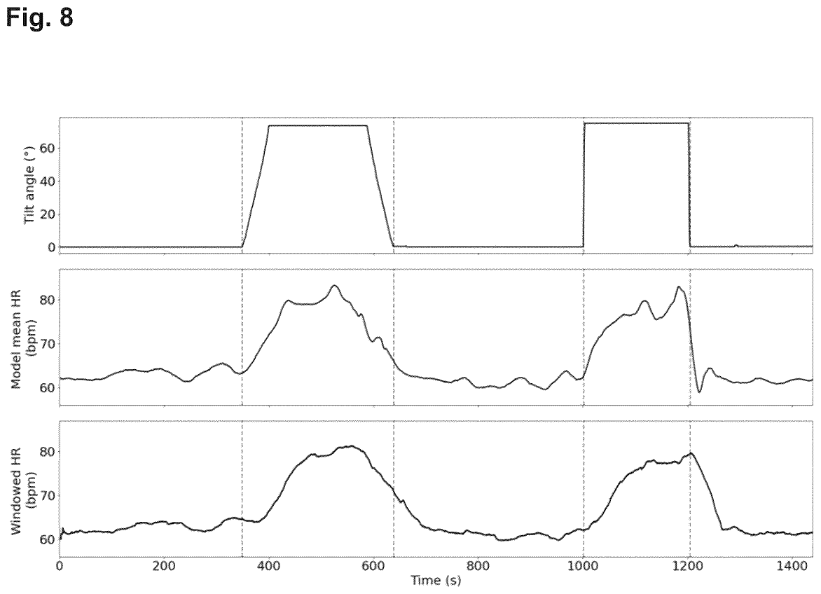

In FIG. 8, we observe the heart rate time series for subject 12726. We compare the results to the clinically-used local averaging method where the heart rate is calculated by taking the average of the inverse of the RR intervals within a window. Specifically, we use a sliding window of 60 s that we slide by 5 ms, as used for a benchmark in R. Barbieri, et al.

We see that μ_{HR} from our algorithm and the standard local averaging method both track the tilt table angle. We are also able to visually differentiate between the slow and rapid tilt in the results from our algorithm as we see μ_{HR} change slower or faster depending on how sudden the tilt is. In contrast, the results of the local averaging method see less of a difference between the two tilts. This is because the windowing procedure has worse time resolution than an SSM and is not able to capture the sudden change in heart rate.

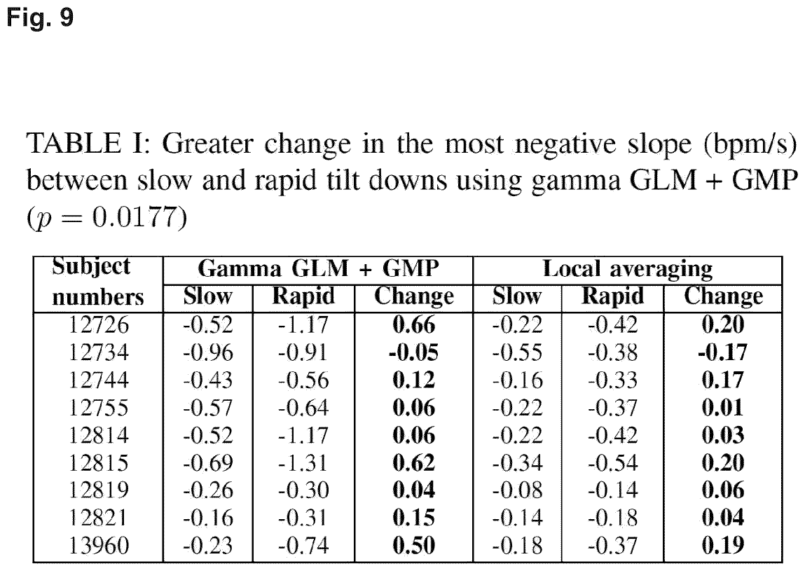

To quantify this difference, we compute the most negative slope in heart rate estimate at the end of the slow and rapid tilts. We do this by obtaining the underlying trend of the heart rate by applying a Savitzky-Golay filter. Then, we use the midpoint formula to approximate the derivative. The difference in slopes between the slow and rapid tilt downs are reported in Table I (FIG. 9). Subject 12734 seemed to have a faster drop in heart rate during the slow tilt than the rapid tilt. For the other 8 subjects, the change in slope was greater in the heart rate estimates from our model in 6 subjects compared to the local averaging method. We are able to see a significantly greater change in rapid vs. slow tilts with our method compared to local averaging (one-tailed paired t-test p=0.0177).

In this example, we present a formulation of heartbeat dynamics that captures time-varying changes by using an SSM with a latent GMP and a gamma GLM measurement model. Compared to preexisting analogous models, this formulation has a few benefits: 1) it fits more naturally to the domain of a GMP, and 2) it has a convex estimation procedure. For 1), the log-link function transforms RR intervals from [symbol] ⁺ to [symbol] , which allows our weights Xₙ to span [symbol] ^{d+1}. The time-varying weights can be used to measure sympathetic and parasympathetic input to the heart. For 2), the convexity of the MAP estimation problem allows us to efficiently estimate the time-varying states using a parallelizable ADMM framework. We also find that our procedure is able to achieve reasonable goodness of fit. In addition, our μ_{HR} estimates visually follow postural changes.

Overall, our work contributes an alternative model of heartbeat dynamics. The convex formulation aids in ease of computation without the need for careful initialization. As our example with heart rate deceleration shows, this framework may be particularly effective compared to alternatives when there is a need to quantify rapid physiological changes in heart rate.

What is claimed is:

1. A computer system, comprising:
a computational processing system; and
memory comprising an application for fitting a point-process model with global optimization;
wherein the application directs the computational processing system to:
receive physiological interval data;
fit the point-process model with global optimization to the physiological interval data; and
compute variability of the physiological interval data using the fitted point-process model with global optimization.

2. The system of claim 1, wherein the physiological interval data is heartbeat interval data.

3. The system of claim 1, wherein the computer system is a part of a health monitoring system that comprises a sensor configured to capture physiological interval data, wherein the application directs the computational processing system to:
sense physiological parameters of a patient to yield the physiological interval data.

4. The system of claim 3, wherein the application directs the computational processing system to perform the following real-time:
sense physiological parameters of a patient to yield the physiological interval data;
receive physiological interval data;
fit the point-process model with global optimization to the physiological interval data; and
compute variability of the physiological interval data using the fitted point-process model with global optimization.

5. The system of claim 3, wherein the sensor is in connection with and the computational processing system is housed within a wearable device, a hemodynamic monitoring system, or an electrocardiography device.

6. The system of claim 3, wherein the sensor is one of: one or more leads of an electrocardiography machine or ECG-like device, a blood pressure transducer catheter, a blood pressure cuff, an ultrasound transducer, a magnetic resonance imaging scanner, or a photoplethysmograph.

7. The system of claim 1, wherein the application directs the computational processing system to:
apply a distribution onto the physiological interval data, wherein the distribution yields a global optimization when utilized with the point-process model.

8. The system of claim 7, wherein the distribution is a gamma distribution.

9. The system of claim 1, wherein the point-process model with global optimization comprises a regression model.

10. The system of claim 9, wherein the regression model is a generalized linear model.

11. The system of claim 1, wherein the application directs the computational processing system to:
infer weights for the physiological interval data.

12. The system of claim 11, wherein the weights are unconstrained and capable of being negative or positive.

13. The system of claim 11, wherein the application directs the computational processing system to:
infer a shape parameter from the weights.

14. The system of claim 11, wherein the memory comprising an application for training a machine-learning model; wherein the application for training a machine-learning model directs the computational processing system to:
train a machine-learning model to predict a biological characteristic from weights inferred for the physiological interval data; wherein the data for training the model comprises inferred weights from at least two cohorts such that the machine-learning model is trained to predict the biological characteristic; wherein the biological characteristic is associated with one of cohort of the at least two cohorts.

15. The system of claim 14, wherein the machine-learning model is to train to predict a likelihood or presence of a medical disorder selected from: orthostatic hypotension, postprandial hypotension, multiple system atrophy, pure autonomic failure, afferent baroreflex failure, familial dysautonomia, heart failure, metabolic disorder, diabetes, or cardiovascular disease.

16. The system of claim 1, wherein the memory comprising an application for fitting a state-space model with global optimization, wherein the application for fitting a state-space model with global optimization directs the computational processing system to:
receive covariate data;
fit the state-space model with global optimization to the physiological interval data and the covariate data; and
compute a relationship between the physiological interval data and the covariate data using the fitted state-space model with global optimization.

17. The system of claim 16, wherein the covariate data is related to the autonomic nervous system.

18. The system of claim 17, wherein the covariate data comprises at least one of: respiration, sleep, stress, posture, inflammation, or drugs.

19. The system of claim 16, wherein the state-space model comprises a Gauss-Markov process.

20. The system of claim 16, wherein the state-space model comprises a high dimensionality solution with an alternating direction method of multipliers.
